(19) United States
(12) Patent Application Publication
(10) Pub. No.: US 2025/0003313 A1
(43) Pub. Date: Jan. 2, 2025

(54) CARBON SEQUESTRATION MONITORING BY MINERAL REACTION EXTENT MONITORING

(71) Applicant: Halliburton Energy Services, Inc., Houston, TX (US)

(72) Inventors: Christopher Michael Jones, Houston, TX (US); Songhua Chen, Katy, TX (US); Ahmed Elsayed Fouda, Pearland, TX (US); Michel Leblanc, Houston, TX (US); Mahmoud Helmy Saada, Houston, TX (US)

(73) Assignee: Halliburton Energy Services, Inc., Houston, TX (US)

(21) Appl. No.: 18/229,361

(22) Filed: Aug. 2, 2023

Related U.S. Application Data
(60) Provisional application No. 63/523,710, filed on Jun. 28, 2023.

(51) Int. Cl. E21B 41/00; E21B 49/00; G01N 33/24; G01V 11/00

(52) U.S. Cl. CPC E21B 41/0064; E21B 49/00; G01N 33/24; G01V 11/002; E21B 2200/20

(57) ABSTRACT

Carbon Capture, Utilization, and Storage (CCUS) is a relatively new technology directed to mitigating climate change by reducing greenhouse gas emissions. Current and new government requirements require proof that carbon dioxide (CO2) is either sequestered in a stable form or safely stored for long periods of time. In instances when the CO2 is sequestered through mineral formation, the need for long-term monitoring can be reduced, as the stability of the sequestered CO2 is inherent based on a chemical change in subterranean rocks. The reactions between CO2 and rock formations are influenced by numerous factors, including temperature, pressure, fluid composition, and the mineralogy of the formation. Furthermore, these reactions occur over large spatial areas and long timescales, making them difficult to monitor directly. Methods and systems of the present disclosure, therefore, may use a combination of laboratory experiments, field monitoring, and modeling to provide convincing evidence of CO2 mineral sequestration.

### CROSS-REFERENCE TO RELATED APPLICATIONS

This application claims benefit to U.S. Provisional Application No. 63/523,710 filed Jun. 28, 2023, which is incorporated herein by reference.

### TECHNICAL FIELD

The present disclosure pertains to identifying changes in subterranean formations based on carbon dioxide (CO2) being injected into a wellbore. More specifically, the present disclosure is directed to sequestering carbon into subterranean formations as effectively and efficiently.

### BACKGROUND

“Carbon Capture, Utilization, and Storage” (CCUS) is a relatively new technology directed to mitigating climate change by reducing greenhouse gas emissions. Governments worldwide have established stringent requirements for carbon storage and sequestration to ensure the long-term safety and effectiveness of CCUS. Typically, these requirements include proof that the stored carbon dioxide (CO2) is either sequestered in a stable form or safely stored for a long period of time that may exceed 100 years.

### BRIEF DESCRIPTION OF THE DRAWINGS

In order to describe the manner in which the features and advantages of this disclosure can be obtained, a more particular description is provided with reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1A is a schematic diagram of an example logging while drilling wellbore operating environment, in accordance with various aspects of the subject technology;

FIG. 1B is a schematic diagram of an example downhole environment having tubulars, in accordance with various aspects of the subject technology;

FIG. 2 illustrates an example of a laboratory setting that may be used to collect data regarding chemical changes that may occur in samples extracted from subterranean formations in the Earth, in accordance with various aspects of the subject technology;

FIG. 3 illustrates several different configurations of apparatus that may be used to collect data that can be analyzed to identify the effectiveness of a carbon sequestration process, in accordance with various aspects of the subject technology;

FIG. 4 illustrates actions that may be performed when a process of carbon sequestration is performed, in accordance with various aspects of the subject technology;

FIG. 5 illustrates actions that may be performed when a laboratory experiment is performed on samples that have been extracted from a wellbore such that operation of a computer model may be improved, in accordance with various aspects of the subject technology; and

FIG. 6 illustrates an example computing device architecture which can be employed to perform various steps, methods, and techniques disclosed herein.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description, or can be learned by practice of the principles disclosed herein. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

As mentioned above, Carbon Capture, Utilization, and Storage (CCUS) is a relatively new technology directed to mitigating climate change by reducing greenhouse gas emissions. Current and new government requirements include proof that the stored carbon dioxide (CO2) is either sequestered in a stable form or safely stored for periods of time that may exceed 100 years. In instances when the CO2 is sequestered through mineral formation, the need for long-term monitoring can be reduced, as the stability of the sequestered CO2 is inherent based on a chemical change in subterranean rocks.

Proving that CO2 has been sequestered as a mineral is, however, a complex task. The reactions between CO2 and rock formations are influenced by numerous factors, including temperature, pressure, fluid composition, and the mineralogy of the formation and the porosity within. Furthermore, these reactions occur over large spatial scales and long timescales, making them difficult to monitor directly. Therefore, a combination of laboratory experiments, field monitoring, and modeling may be required to provide convincing evidence of CO2 mineral sequestration.

Laboratory experiments on whole core, core plug samples or drill cuttings extracted from subterranean formations can provide valuable data on the potential for chemically binding CO2 to rocks or minerals (CO2 mineralization) in the Earth. These experiments can involve exposing extracted core samples or cuttings to CO2 under controlled conditions while monitoring changes in their mineralogy, fluid composition, and physical properties in the laboratory. Techniques such as X-ray diffraction, scanning electron microscopy, and energy-dispersive X-ray spectroscopy can be used to identify and quantify the minerals present. Changes in the composition of the brine in the cores can provide indirect evidence of mineral trapping. Additionally, Nuclear Magnetic Resonance (NMR) or electromagnetic sensing elements can be used to monitor changes in the porosity and permeability of the cores, which can indicate the formation of new minerals within the pore spaces of the rock. Moreover, acoustic sensors can be used to monitor changes the elasticity and geo-mechanical property changes as the solidification of carbonized mineral formation changes rock's acoustic responses. Furthermore, temperature-accelerated studies can be conducted to estimate reaction rates under reservoir conditions, and calorimetry can be used to measure the heat produced by the reactions, providing insights into their thermodynamics for long time sequestration through mineralization predictions.

In a field of wellbores, various monitoring techniques can be used to track changes in Earth formations over time. These include geophysical logging tools that can detect changes in the formation's mineralogy, resistivity, temperature, and physical properties. Fiber optic cables can provide high-resolution temperature data over the entire length of the well, while resistivity tools can provide information about the formation several tens to hundreds of feet away from the wellbore. Changes in these parameters can indicate the occurrence and extent of CO2 mineralization reactions. Additionally, Nuclear Magnetic Resonance (NMR) logging tools can be used to monitor changes in the porosity and permeability of subterranean formations, which can provide further evidence of CO2 mineralization. These tools can provide information on the distribution of fluids in the pore spaces of the rock and can help track the movement and fate of CO2 after that CO2 has been injected into an Earth formation.

Described herein are systems, apparatuses, processes (also referred to as methods), and computer-readable media (collectively referred to as “systems and techniques”) for improving an accuracy of determinations made using data sensed in a wellbore. By correlating the results of laboratory experiments with field monitoring data, techniques of the present disclosure may be used to build a compelling case for CO2 mineral sequestration. For example, if certain minerals are found to react with CO2 in the laboratory experiments, and changes in temperature or resistivity consistent with these reactions are observed in a set of wellbores, such data would provide strong evidence of CO2 mineralization. Furthermore, by using geochemical modeling to predict the reactions between CO2, brine, and the formation minerals, these predictions can be compared with the results of the monitoring efforts to validate the model and provide additional evidence of mineral trapping.

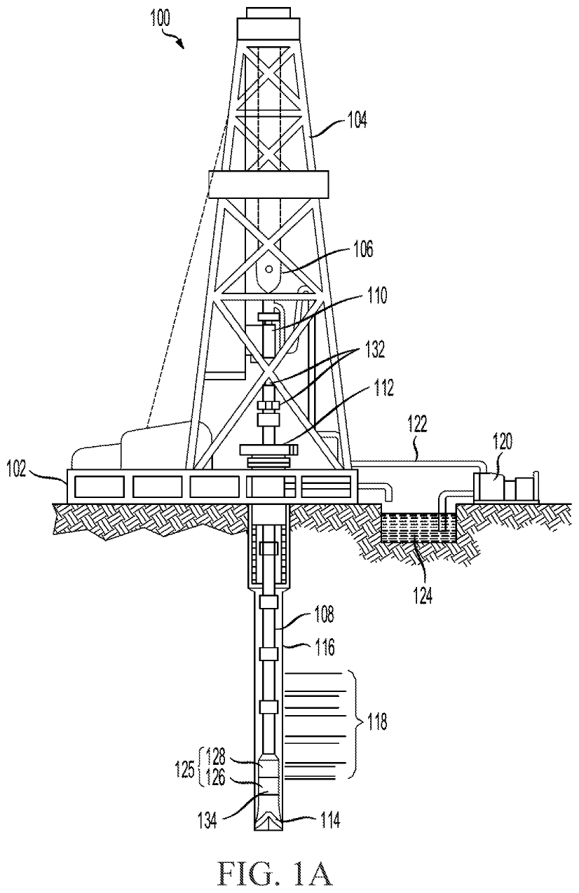

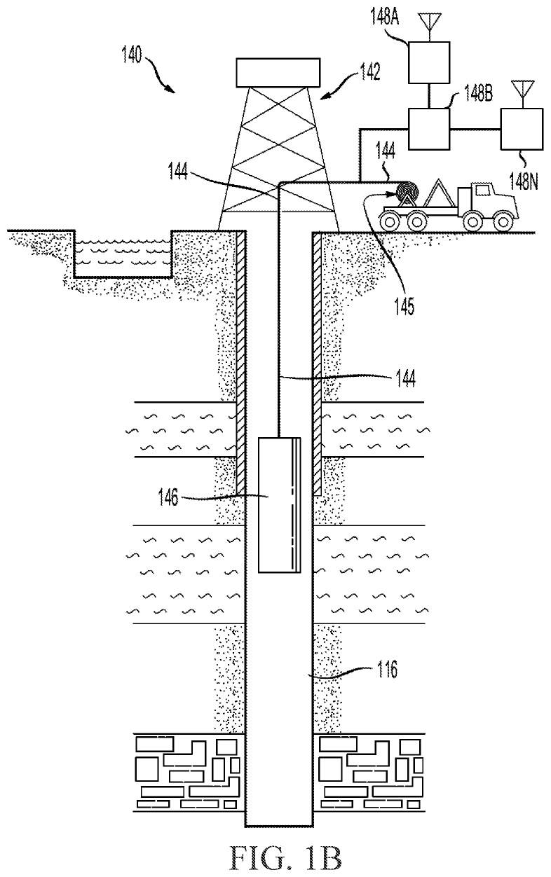

Turning now to FIG. 1A, a drilling arrangement is shown that exemplifies a Logging While Drilling (commonly abbreviated as LWD) configuration in a wellbore drilling scenario 100. Logging-While-Drilling typically incorporates sensors that acquire formation data. Specifically, the drilling arrangement shown in FIG. 1A can be used to gather formation data through an electromagnetic imager tool as part of logging the wellbore using the electromagnetic imager tool. The drilling arrangement of FIG. 1A also exemplifies what is referred to as Measurement While Drilling (commonly abbreviated as MWD) which utilizes sensors to acquire data from which the wellbore's path and position in three-dimensional space can be determined. FIG. 1A shows a drilling platform 102 equipped with a derrick 104 that supports a hoist 106 for raising and lowering a drill string 108. The hoist 106 suspends a top drive 110 suitable for rotating and lowering the drill string 108 through a well head 112. A drill bit 114 can be connected to the lower end of the drill string 108. As the drill bit 114 rotates, it creates a wellbore 116 that passes through various subterranean formations 118. A pump 120 circulates drilling fluid through a supply pipe 122 to top drive 110, down through the interior of drill string 108 and out orifices in drill bit 114 into the wellbore. The drilling fluid returns to the surface via the annulus around drill string 108, and into a retention pit 124. The drilling fluid transports cuttings from the wellbore 116 into the retention pit 124 and the drilling fluid's presence in the annulus aids in maintaining the integrity of the wellbore 116. Various materials can be used for drilling fluid, including oil-based fluids and water-based fluids.

Logging tools 126 can be integrated into the bottom-hole assembly 125 near the drill bit 114. As the both drill bit 114 extends into the wellbore 116 through the formations 118 and as the drill string 108 is pulled out of the wellbore 116, logging tools 126 collect measurements relating to various formation properties as well as the orientation of the tool and various other drilling conditions. The logging tool 126 can be applicable tools for collecting measurements in a drilling scenario, such as the electromagnetic imager tools described herein. Each of the logging tools 126 may include one or more tool components spaced apart from each other and communicatively coupled by one or more wires and/or other communication arrangement. The logging tools 126 may also include one or more computing devices communicatively coupled with one or more of the tool components. The one or more computing devices may be configured to control or monitor a performance of the tool, process logging data, and/or carry out one or more aspects of the methods and processes of the present disclosure.

The bottom-hole assembly 125 may also include a telemetry sub 128 to transfer measurement data to a surface receiver 132 and to receive commands from the surface. In at least some cases, the telemetry sub 128 communicates with a surface receiver 132 by wireless signal transmission. e.g, using mud pulse telemetry, EM telemetry, or acoustic telemetry. In other cases, one or more of the logging tools 126 may communicate with a surface receiver 132 by a wire, such as wired drill pipe. In some instances, the telemetry sub 128 does not communicate with the surface, but rather stores logging data for later retrieval at the surface when the logging assembly is recovered. In at least some cases, one or more of the logging tools 126 may receive electrical power from a wire that extends to the surface, including wires extending through a wired drill pipe. In other cases, power is provided from one or more batteries or via power generated downhole.

Collar 134 is a frequent component of a drill string 108 and generally resembles a very thick-walled cylindrical pipe, typically with threaded ends and a hollow core for the conveyance of drilling fluid. Multiple collars 134 can be included in the drill string 108 and are constructed and intended to be heavy to apply weight on the drill bit 114 to assist the drilling process. Because of the thickness of the collar's wall, pocket-type cutouts or other type recesses can be provided into the collar's wall without negatively impacting the integrity (strength, rigidity and the like) of the collar as a component of the drill string 108.

Referring to FIG. 1B, an example system 140 is depicted for conducting downhole measurements after at least a portion of a wellbore has been drilled and the drill string removed from the well. An electromagnetic imager tool can be operated in the example system 140 shown in FIG. 1B to log the wellbore. A downhole tool is shown having a tool body 146 in order to carry out logging and/or other operations. For example, instead of using the drill string 108 of FIG. 1A to lower the downhole tool, which can contain sensors and/or other instrumentation for detecting and logging nearby characteristics and conditions of the wellbore 116 and surrounding formations, a wireline conveyance 144 can be used. The tool body 146 can be lowered into the wellbore 116 by wireline conveyance 144. The wireline conveyance 144 can be anchored in the drill rig 142 or by a portable means such as a truck 145. The wireline conveyance 144 can include one or more wires, slicklines, cables, and/or the like, as well as tubular conveyances such as coiled tubing, joint tubing, or other tubulars. The downhole tool can include an applicable tool for collecting measurements in a drilling scenario, such as the electromagnetic imager tools described herein.

The illustrated wireline conveyance 144 provides power and support for the tool, as well as enabling communication between data processors 148A-N on the surface. In some examples, wireline conveyance 144 can include electrical and/or fiber optic cabling for carrying out communications. The wireline conveyance 144 is sufficiently strong and flexible to tether the tool body 146 through the wellbore 116, while also permitting communication through the wireline conveyance 144 to one or more of the processors 148A-N, which can include local and/or remote processors. The processors 148A-N can be integrated as part of an applicable computing system, such as the computing device architectures described herein. Moreover, power can be supplied via wireline conveyance 144 to meet power requirements of the tool. For slickline or coiled tubing configurations, power can be supplied downhole with a battery or via a downhole generator.

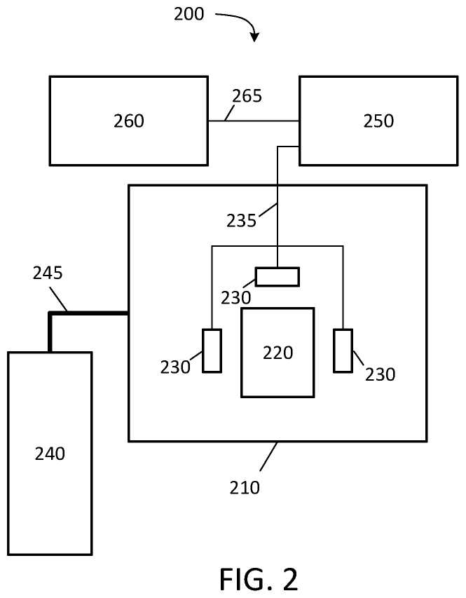

FIG. 2 illustrates an example of a laboratory setting that may be used to collect data regarding chemical changes that may occur in samples extracted from subterranean formations in the Earth. The laboratory test setting 200 of FIG. 2 includes chamber 210 that contains sample 220 and sensors 230. Test setting 200 also includes fluid tank 240, computer 250, and spectrographic device 260. Sensors 230 are communicatively coupled to computer 250 via bus 235. Bus 235 may also be used to provide power to sensors 230. Fluid tank 240 is coupled to chamber 210 via pipe 245. Bus 265 may be used to communicatively couple to computer 250.

Sample 220 may be a core sample, or a cutting taken from a wellbore. As such, sample 220 represents materials that may be found in strata of the Earth proximal to a wellbore that is being developed or that has been developed. Sensors 230 may be any type of sensors or sensing apparatus known in the art. As such, sensors 230 may be temperature sensors, electromagnetic (EM) sensors, sensing elements of a nuclear magnetic resonance (NMR) sensing device, acoustic/seismic sensors, or sensors associated with any of an X-ray diffraction device, a scanning electron microscope, an X-ray spectrometer, or other device.

Chamber 210 may be a pressure-temperature chamber capable of containing pressures and being heated to temperatures that are consistent with pressures and temperatures found in subterranean environments. As such chamber 210 may include pressure seals and a heating element that allow the temperature and pressure inside of chamber 210 to be controlled, for example, by computer 250. Fluid tank 240 may provide fluid (e.g., gaseous or liquid CO2) to chamber 210 when laboratory experiments are performed. While not illustrated in FIG. 2, a pressure control device may be attached to pipe 245 such that fluids from fluid tank can be controlled.

In operation, a core sample or cuttings (sample 220) may be placed in chamber 210 after sample 220 has been extracted from the Earth. Sensors 230 may be arranged around sample 220 and chamber 210 may be sealed. The temperature of chamber 210 may be heated to a temperature likely to be encountered in a subterranean formation and a fluid may be provided to chamber 240. A controlled mass of the fluid (e.g., gaseous or liquid CO2) may be provided to chamber 210 during an experiment. While not illustrated in FIG. 2, other materials may be provided to chamber 210. These other materials may be substances that are found in subterranean formations that may act to accelerate or mitigate chemical changes that may occur in sample 220 when sample 220 is exposed to CO2 at pressures and temperatures that may be found within the Earth. For example, since water and hydrocarbons are often found in subterranean formations, concentrations of water and/or hydrocarbons may be provided to chamber 210 when effects on sample 220 in an environment that contains concentrations of water, hydrocarbons, and/or CO2 are evaluated.

In certain instances, after sample 220 is arranged in chamber 210, an experimental environment may be arranged to correspond to an initial wellbore condition. The initial wellbore condition may correspond to a temperature and a pressure common to a particular depth within the Earth. When conditions within chamber 210 match to a threshold degree the initial wellbore condition, a heater that heats chamber 210 may be turned off. Carbon dioxide (CO2) may then be provided to chamber 210 in a manner that simulates CO2 being injected into a subterranean environment proximal to a wellbore. Sensors 230 may then monitor conditions within the chamber. Changes in temperature or CO2 concentrations may be monitored and these changes may be used to identify whether chemical changes appear to be occurring within sample 220. Additionally, sensors 230 may be used to collect NMR data, electromagnetic (EM) data, X-ray data, or other data and provide this data to computer 250. Computer 250 may then make evaluations to validate the extent of chemical changes in sample 220. Sensors 230 may be similar to sensors that are commonly deployed in wellbores when strata next to those wellbores are evaluated. As such, data from sensor 230 and from other evaluations may be used to improve, update, or train computer models that estimate amounts of CO2 that has been converted into minerals at locations where CO2 may be sequestered in the Earth.

An individual experiment could be run of a span of time (e.g., hours, days, weeks, or other time span) that is sufficient for some possible chemical reactions in sample 220 to occur. During this time span, additional CO2 may be provided to chamber 210. Furthermore, a heater at chamber 210 may be turned on or off depending on constraints of the experiment. While not mentioned above, sample 220 or portions (e.g., shavings or dissolved pieces) of sample 220 may be placed into spectrographic device 260 to collect information about chemical properties of sample 220. After sample 220 has been in chamber 210 for the span of time, sample 220 may be removed from chamber 210 such that additional evaluations may be performed. This may include, placing sample 220 or portions (e.g., new shavings or dissolved pieces) of sample 220 into spectrographic device 260. Spectrographic device 260 may perform tests such that chemical contents of sample 220 may be determined. Data from spectrographic device 260 may be provided to computer 250 via communication bus 265.

Data collected by spectrographic device 260 before sample 220 was placed into chamber 220 may be compared to data collected by spectrographic device 260 after sample 220 has been exposed to the simulated wellbore conditions for the span of time. Data collected by comparing before and after data from the spectrographic device 260 combined with data collected by sensors 230 may be used to improve, adjust, or train the computer models that simulate chemical changes in rocks located at locations where the CO2 may be sequestered in the Earth.

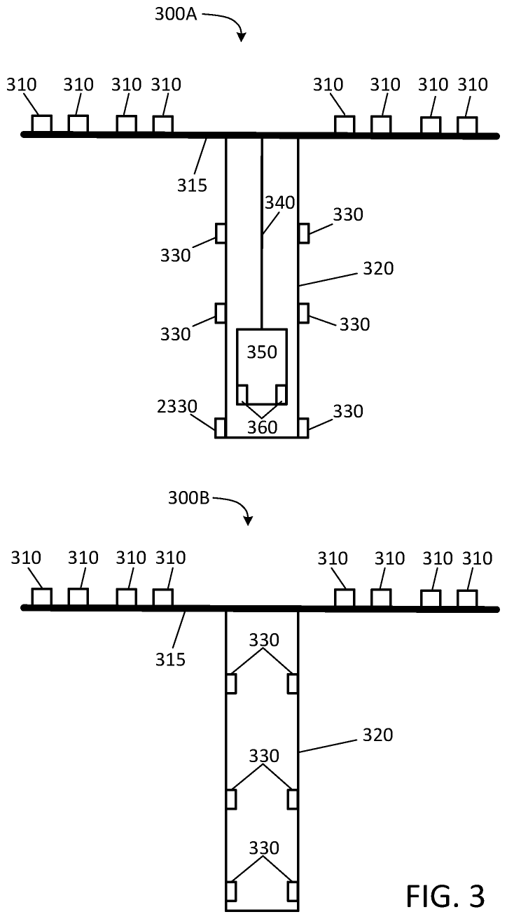

FIG. 3 illustrates several different configurations of apparatus that may be used to collect data that can be analyzed to identify the effectiveness of a carbon sequestration process. FIG. 3 includes a first wellbore measurement configuration 300A and a second wellbore measurement configuration 300B. Each of these wellbore configurations 300A and 300B includes wellbore or casing 320, elements 310, and elements or sensors 330. Wellbore configuration 300A also includes sensing apparatus 350 that may have been lowered into wellbore or casing 320 using wireline conveyance 340. Wireline conveyance 340 may be used to deploy sensing apparatus 350 in a way that is similar to the deployment of tool body 146 discussed in respect to FIG. 1B. Sensing apparatus 350 may also include elements or sensors 360.

In one instance, sensing apparatus 350 may be lowered into wellbore 320 before a casing has been added to the wellbore 320. At this time sensing apparatus 350 may collect data that may be used to characterize rock formations or types of materials that are located next to wellbore 320. Elements or sensors 360 of sensing apparatus 350 may collect data of any sort (electromagnetic data, X-ray data, acoustic data, NMR data, or other) such that an initial evaluation rock formations and materials may be performed. A casing may be deployed in wellbore 320. This may include attaching sensors 330 to an outside of the casing.

In another instance, as illustrated in measurement configuration 300B, elements or sensors 330 may be deployed on an inside surface of wellbore or casing 320. These sensors may be used to monitor conditions of the wellbore environment. When a casing is used, that casing may be made of materials (e.g., a non-metallic material like fiberglass) that do not or that minimally attenuate fields (e.g., electromagnetic fields or fields used by an NMR or other sensor) of a measurement device. By placing elements or sensors 330 on an inside surface of such a casing or wellbore 320, the likelihood of damaging these elements or sensors 330 may be mitigated. By using a casing made of materials that do not or that minimally attenuate measurement fields, measurements may be made through the casing while the casing helps maintain structural stability of the wellbore. By using a casing made from an electrically insulating material such as fiberglass, casings used in wellbores that sequester carbon will resist corrosive (acidic) effects of brine generated from adding CO2 to the wellbore. Such electrical insulating casings will also allow electromagnetic sensing and NMR sensing devices to operate more effectively because the electrical insulating casing will allow electromagnetic fields used by electromagnetic sensing devices and RF signals used by NMR sensing devices to more readily pass through the casing as compared to casings that are made of materials like steel.

In each configuration 300A and 300B, elements 310 are disposed along the Earth's surface 315. Elements 310 may be used to transmit energy (e.g., electromagnetic or acoustic energy) into the Earth. Portions of this transmitted energy may then be sensed by elements or sensors 330. Data received by elements or sensors 330 may be provided to a computer that analyzes this received data. Evaluations performed by the computer may be used to identify changes in strata of the Earth when a carbon sequestration process is performed. These evaluations may identify whether CO2 provided to the wellbore is present in a liquid form, a gaseous form, or may identify a mass of carbon in the CO2 that has been converted into a mineral form. Alternatively or additionally, elements or sensors 330 may be used to transmit energy that is received by other elements or sensors 330 or by elements 310.

Data collected by sensors that sense electromagnetic (EM) energy may be used to identify the resistivity of areas within the Earth. When CO2 is injected into formations within the Earth, the resistivity of areas within those formations change with concentrations of CO2. This is because the resistivity of fluids and rock included in subterranean formations are different from the resistivity of CO2. As such, measurements of EM energy may be used to identify where a plume of injected CO2 migrates to in those formations based on changes in resistivity and contrasts between resistivity. Alternatively or additionally, acoustic or seismic measurements may be used to identify areas where the CO2 has migrated. Acoustic or seismic devices may transmit acoustic energy and portions of that acoustic energy may be received by wellbore sensors such that evaluations may be made.

Nuclear magnetic resonance (NMR) sensors may also be deployed in a wellbore to collect data from which determinations may be made regarding an extent of chemical reactions that have occurred in rocks that are near the wellbore. NMR data collected before CO2 has been injected into the subterranean formations may be compared with NMR data collected after the CO2 is injected to identify changes in roughness, porosity, and/or permeability of rocks near the wellbore.

Data collected using various different types of measurements (e.g., EM, acoustic/seismic, NMR, and/or temperature) could be evaluated to identify an extent of chemical reaction. Temperature changes may be used to identify an amount of heat released by a chemical reaction as most chemical reactions that convert CO2 into mineralized carbon compounds are exothermic (i.e., generate heat). Computer models that use different types of data (EM data, NMR data, acoustic/seismic data, laboratory data, and/or temperature change data) may more accurately identify rates of mineral formation. Such computer models may be used to identify chemical reaction rates based on a total amount of heat produced, and/or based on changes in resistivity, density, roughness, porosity, and permeability identified using one or more different types of measurements. These computer models may also model the heat capacity and/or the thermal conductivity of rock, CO2, and fluids located in rock formations.

Once a mass of CO2 has been injected into a subterranean formation and chemical reactions have begun. The computer model may be used to estimate portions of the mass of CO2 that should be mineralized in the future. As such, computer models may be able to identify an extent of current mineralization and then be used to forecast future extents of mineralization.

Since metal casings (e.g., steel casings) will tend to block electromagnetic fields as meatal casings will act as a Faraday shield, casings that do not include metal may be used. As mentioned above, fiberglass casings may be used. Since fiberglass does not include metal, it will not significantly block the transmission of electrical or magnetic fields used by EM sensing devices or NMR sensing devices. The use of non-metallic casings along at least portions of the wellbore may allow sensors that are deployed underground to be deployed within a casing that physically isolates or protects those sensors from rocks located near the wellbore.

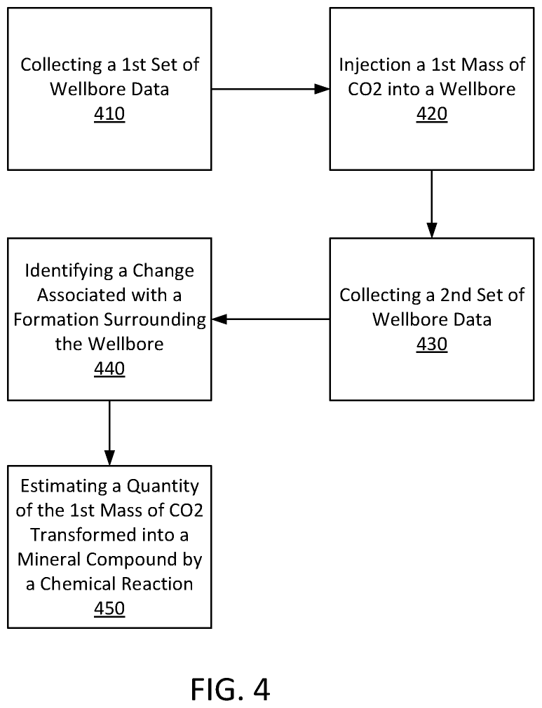

FIG. 4 illustrates actions that may be performed when a process of carbon sequestration is performed. At block 410, a first set of wellbore data may be collected. This first set of wellbore data may include temperature data, pressure data, EM sensor data, acoustic/seismic data, and/or NMR sensor data that was collected before a first mass of CO2 is injected into formations that surround a wellbore. At block 420 the first mass of CO2 may be injected into the formations that surround the wellbore. As the CO2 is injected into the wellbore formations, additional data may be collected and one or more processors executing instructions of a computer model may identify how a plume of CO2 migrates into the wellbore formations. This may include evaluating EM data and/or acoustic/seismic data to identify changes in resistivity or density in the wellbore formations. Over time, chemical reactions that convert portions of the CO2 into minerals may occur and this may result in the temperature within the wellbore and wellbore structures increasing. As mentioned above, data collected by different types of sensors may be used to identify the movement of CO2 as well as chemical reaction rates based on a total amount of heat produced, and/or based on changes in resistivity, density, roughness, porosity, and permeability identified using one or more different types of measurement based on operation of a computer model. As such the one or more processors may execute the instructions of the computer model to identify changes that occur within subterranean formations over time.

At block 430 a second set of wellbore data may be collected. This second set of data may be collected after some percentage of the CO2 provided to the wellbore has been transformed into a mineral compound by a chemical reaction. At block 440, evaluations performed by one or more processors may identify a change associated with the formation that surrounds the wellbore. The evaluations performed by the processors may estimate a quantity of the first mass of CO2 that has been transformed into the mineral compound by the chemical reaction at block 450.

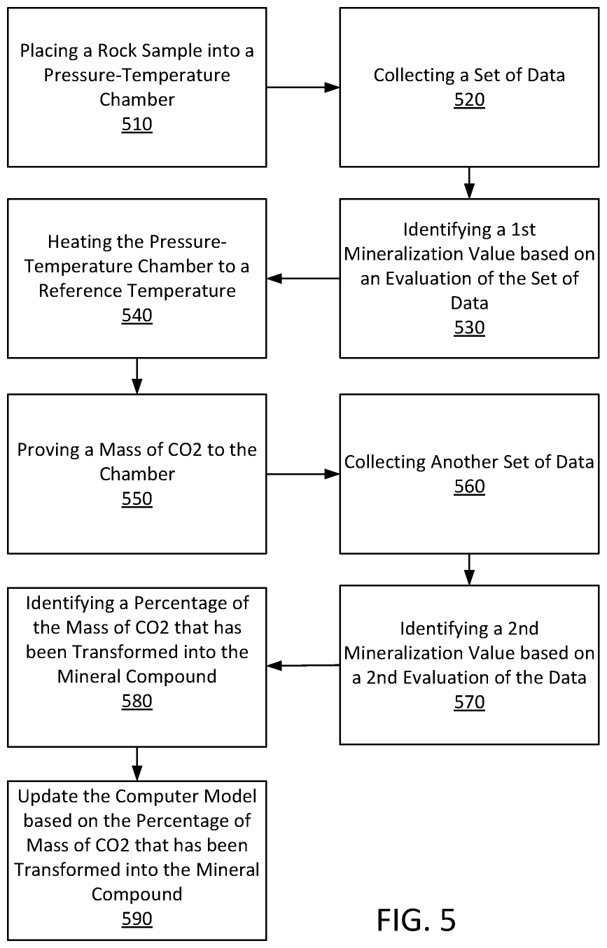

FIG. 5 illustrates actions that may be performed when a laboratory experiment is performed on samples that have been extracted from a wellbore such that operation of a computer model may be improved. At block 510, a rock sample may be placed into a pressure-temperature chamber as discussed in respect to FIG. 2. As noted above, examples of a rock sample include a core sample or cuttings extracted from a wellbore. Operations performed at block 510 may include arranging sensors in proximity to the sample and sealing a door of the pressure-temperature chamber. A first set of data may then be collected at block 520. This first set of data may include EM data, acoustic/seismic data, and/or NMR data from which characteristics of the sample may be identified. Such characteristics may include density, porosity, permeability, fluid content, and/or a mineralization value of the sample.

At block 540, the pressure-temperature chamber may be heated to a reference temperature. This reference temperature may correspond to a temperature that may be found in Earth formations where CO2 will be sequestered. The pressure of the chamber may also be controlled by providing a fluid to the pressure-temperature chamber. The fluid provided to the chamber may include a mass of CO2. At block 550 the mass of CO2 may be provided to the chamber. The CO2 may be provided to the chamber while additional sensor data is collected. At block 560 another set of data sensor data may be collected. This other set of sensor data may be evaluated at block 570 to identify a second mineralization value. From this second mineralization value, a percentage of the mass of CO2 provided to the chamber that has been transformed into the mineral compound may be identified. This percentage of CO2 that is transformed may be identified based on observed temperature changes and/or based on changes in EM data, NMR data.

Some portions of the data collected in the laboratory setting may be collected using the same types of measurements used to collect data in a wellbore setting, where other portions of the data collected in the laboratory setting may be collected using types of measurements (e.g., physical measurements) that cannot be performed in the wellbore setting. For example, in the laboratory setting, the weight of the sample may be measured before and after an experiment to identify how much carbon was absorbed by the sample. Fluids may be provided to the sample in controlled ways to measure porosity and/or permeability of the sample before and after the experiment. Portions of the sample may be provided to a spectrographic device (e.g., spectrographic device 260 of FIG. 2) to identify chemical compositions of the sample before and after the experiment.

One or more processors executing instructions of the computer model may use data collected using types of sensors that are commonly used in a wellbore environment and these processors may identify an estimate of the mass carbon of the CO2 that has been transformed into the mineral compound at block 580. This estimate may be compared to masses of carbon from the CO2 that has been transformed into the mineral compound using laboratory measurements (the physical measurements). When a measured mass of the carbon transformed into the minerals does not correspond to a threshold degree with the estimated mass of carbon of the CO2 that has been transformed into the mineral compound, the computer model may be updated. The computer model may be updated at block 590 based on the percentage of mass of the CO2 that has been transformed into the mineral compound.

One type of formation that CO2 may be sequestered into may be classified as siliciclastic reservoirs. Siliciclastic reservoirs are primarily composed of silicate minerals, and carbonate reservoirs, largely made up of carbonate minerals, each present unique opportunities and challenges for CO2 mineral sequestration. Siliciclastic reservoirs are typically dominated by quartz, feldspars, and clay minerals. Quartz is relatively inert, but feldspars and clay minerals can react with CO2 to form carbonate minerals and silica. For example, the reaction of potassium feldspar (KAlSi3O8) with CO2 and water can produce kaolinite (a type of clay), potassium ions, and bicarbonate ions. Clay minerals, such as smectite, can also react with CO2 and water to form illite and carbonate minerals.

The reactions of CO2 with these minerals can cause significant changes in the composition of the brine in the reservoir. For example, the reaction of feldspar or clay minerals with CO2 can increase the concentration of bicarbonate ions in the brine, making the brine more alkaline. The dissolution of carbonate minerals can increase the concentrations of calcium and magnesium ions in the brine. Over time, these changes in brine composition can provide indirect evidence of CO2 mineralization.

Another type of formation where CO2 may be sequestered includes mafic minerals that are formed from volcanic or magma processes. Mafic minerals can be found in purely mafic formations or transported to siliciclastic formations. Mafic minerals may offer the highest potential to capture CO2. Mafic minerals, which are rich in magnesium and iron, can also react with CO2 to form carbonate minerals. These reactions are particularly important in the context of CO2 sequestration because they can permanently lock away CO2 in a stable, solid form. Below some examples of these reactions:

Olivine: Olivine (Fe,Mg)2SiO4 is a common mafic mineral that can react with CO2 to form serpentine and magnetite. This reaction also produces silica and releases iron and magnesium ions into the brine. The reaction can be represented as follows: (Fe,Mg)2SiO4+CO2→Mg3Si2O5(OH)4+Fe3O4+SiO2+Mg2++Fe2+

Pyroxene: Pyroxene ((Fe,Mg)SiO3) is another mafic mineral that can react with CO2 to form carbonate minerals. This reaction also produces silica and releases iron and magnesium ions into the brine. The reaction can be represented as follows: (Fe,Mg)SiO3+CO2→MgCO3+FeCO3+SiO2+Mg2+++Fe2+

Amphibole: Amphibole is a group of mafic minerals that can also react with CO2 to form carbonate minerals. These reactions are more complex and can produce a variety of products, including silica, serpentine, and talc, as well as releasing various ions into the brine.

These reactions can significantly change the composition of the brine in the reservoir. The formation of carbonate minerals can increase the alkalinity of the brine, while the release of iron and magnesium ions can increase its hardness. Over time, these changes in brine composition can provide indirect evidence of CO2 mineralization. However, they can also affect the physical and chemical properties of the reservoir, such as its porosity, permeability, which may be monitored by NMR in order to monitor the reaction progress.

Fiber optic temperature monitoring can play a crucial role in tracking the extent of mineralization reactions during CO2 sequestration. As these reactions are generally exothermic, they release heat that can be detected as a temperature increase in the surrounding formation. Fiber optic cables, when deployed along the length of the well, can provide high-resolution temperature data, allowing for the detection of localized hot spots where reactions may be occurring. By correlating these temperature increases with the known enthalpy changes for specific mineralization reactions, it's possible to estimate the extent of the reactions and, consequently, the amount of CO2 that has been sequestered. However, interpreting this temperature data requires a comprehensive heat flow model that can account for various factors such as the thermal conductivity and heat capacity of the formation, the temperature and pressure conditions, and the rate of CO2 injection. Core measurements can provide valuable data for developing and refining this heat flow model. For example, laboratory experiments on core samples can be used to measure the heat produced by the reactions under controlled conditions, providing a direct link between the observed temperature changes and the extent of CO2 mineralization.

Monitoring the composition of the brine, either through direct analysis or resistivity measurements, can provide valuable constraints for the heat production model used in CO2 sequestration. As CO2 reacts with the minerals in the formation, it can cause changes in the concentrations of various ions in the brine as noted above. These changes can be detected either by analyzing the brine samples directly or by observing changes in the resistivity of the brine, as the resistivity is sensitive to the ion concentration. By correlating these changes in brine composition with the temperature data from the fiber optic monitoring, it's possible to refine the heat flow model. For example, if certain ions are increasing or decreasing in concentration at the same time that the temperature is changing, it could indicate that those ions are involved in exothermic (or endothermic) reactions with CO2. This information can help to better predict the heat produced by the reactions and thus improve the accuracy of the heat flow model.

In addition to constraining the heat production model, monitoring the brine composition can also provide an indirect way to monitor the extent of the CO2 mineralization reactions. The reactions between CO2 and the minerals in the formation can produce specific ions that are released into the brine. By tracking the concentrations of these ions over time, it's possible to estimate the extent of the reactions and the amount of CO2 that has been sequestered. For example, a decrease in calcium or magnesium ions and an increase in bicarbonate ions could suggest the formation of carbonate minerals. Therefore, regular monitoring of the brine composition, either through direct sampling or resistivity measurements, can provide valuable insights into the progress of CO2 sequestration.

Nuclear Magnetic Resonance (NMR) logging is a powerful tool that can provide valuable insights into the changes in a formation during CO2 sequestration. NMR works by measuring the response of hydrogen nuclei (protons) in the formation's fluids to a magnetic field, which can provide information about the size and connectivity of the pore spaces in the rock. As CO2 reacts with the minerals in the formation to form new minerals, it can cause changes in the porosity and permeability of the rock, which can be detected by NMR. For example, the formation of new minerals within the pore spaces can decrease the porosity and permeability, while the dissolution of minerals can increase them. NMR can also provide information about the surface roughness of the pore spaces, as the relaxation time of the protons is influenced by the surface-to-volume ratio and surface relaxivity of the pores. Therefore, by monitoring changes in the NMR response over time, it's possible to track the progress of the CO2 mineralization reactions and their impact on the physical properties of the formation.

The natural variation in mineral content within a set of cores taken along the length of the wellbore can provide a valuable window into the range of potential reactions that can occur within the reservoir during CO2 sequestration. Each core sample, with its unique mineral composition, represents a different microenvironment where a distinct set of reactions may take place. By studying these reactions in the laboratory under controlled conditions, it's possible to develop a composite picture of the reactions that can occur throughout the reservoir. This composite set of reactions can then be used to interpret the field monitoring data. For example, changes in brine composition, temperature, and the physical properties of the formation (such as porosity, permeability, and surface roughness) can be compared with the expected outcomes of the composite reactions to infer which reactions are likely occurring. This approach, known as inverse modeling, can provide a powerful tool for tracking the progress of CO2 mineralization and for predicting the long-term behavior of the sequestered CO2. By integrating laboratory experiments, field monitoring, and modeling, it's possible to build a comprehensive understanding of the CO2 sequestration process and to provide the evidence needed to demonstrate the effectiveness and safety of this critical climate mitigation technology.

The process of drilling and retrieving core samples can cause physical damage to the rock, which can potentially affect the results of laboratory experiments. This damage can include microfractures, changes in porosity and permeability, and alterations in the mineral surface properties, all of which can influence the reactions between CO2 and the minerals in the rock. Therefore, it's crucial to mitigate and account for this drilling damage in order to obtain reliable laboratory data. This can involve careful handling and preservation of the cores, as well as the use of non-destructive testing methods to assess the extent of the damage. The drilling damage may once assessed may be correlated to the experimental results and experimental results extrapolated to a result as if there were no damage. Additionally, it's important to consider the potential impact of drilling damage when interpreting the results of the experiment. For example, if a core sample shows unusually high reactivity, it could be due to increased surface area from microfractures rather than the inherent reactivity of the minerals. Other times data must be excluded from the experimental results or at least qualitatively interpreted. By taking these factors into account, it's possible to obtain more accurate and meaningful data from the core experiments, which can improve the understanding of CO2 mineralization and enhance the reliability of the evidence for CO2 sequestration.

Core damage during drilling can affect the results of laboratory experiments. The damage can alter the physical properties of the core, such as its porosity and permeability, and can also expose fresh mineral surfaces that may react differently with CO2. Techniques to mitigate the effect of damage on experiments include:

Use Multiple Cores: Using multiple core samples can help to account for heterogeneity in the formation and variability in core damage. By comparing the results from different cores, it is possible to identify trends or patterns that are consistent across all samples, and thus likely to be representative of the formation as a whole.

Core Preservation: Minimizing core damage during drilling and handling is crucial. Using appropriate drilling fluids, minimizing exposure to air, and storing the cores under conditions similar to those in the formation (e.g., under pressure and saturated with brine) can help to preserve their original properties.

Core Analysis: Detailed analysis of the cores before the experiments can provide information on the extent of damage. Techniques such as computed tomography (CT) scanning, SEM imaging, and porosity/permeability measurements can reveal physical alterations in the core. Chemical analysis (e.g., XRD or EDS) can identify changes in mineralogy that might result from exposure to drilling fluids or air.

Damage Assessment: Performing a baseline set of measurements before and after the CO2 injection experiment can help quantify the extent of any changes due to the experiment itself. This could include physical measurements (e.g., porosity, permeability) and chemical analyses (e.g., mineralogy, brine composition).

Modeling: Geochemical modeling can help interpret the experimental results. By incorporating parameters such as the surface area of reactive minerals and the extent of core damage into the model, it is possible to estimate how these factors are affecting the CO2 mineralization reactions.

Sidewall Cores: If core damage is a significant concern, then taking sidewall cores in addition to conventional cores. Sidewall cores are smaller and taken from the side of the borehole, which can sometimes result in less damage to a sample.

The use of fiberglass casing in a wellbore can significantly enhance the ability to conduct long-term monitoring of CO2 sequestration. Unlike traditional metal casings, fiberglass casings are transparent to electromagnetic waves, which enables the use of deep resistivity or dielectric logging tools to monitor changes in the formation far away from the wellbore. These tools can provide valuable information about the movement of the CO2 plume and changes in the brine composition, which can help track the progress of CO2 mineralization. In addition, fiberglass casings allow for the use of Nuclear Magnetic Resonance (NMR) logging tools, which can provide information about the porosity, permeability, and fluid content of the formation. This can provide further evidence of CO2 mineralization, as the formation of new minerals can cause changes in these properties. Furthermore, fiberglass casings provide better thermal isolation than metal casings, which can simplify the heat flow model used to interpret temperature data from fiber optic monitoring. By reducing the influence of heat transfer between the wellbore and the formation, fiberglass casings can make it easier to detect the heat produced by the CO2 mineralization reactions. Therefore, the use of fiberglass casing can provide a range of benefits for monitoring CO2 sequestration, enhancing the reliability of the monitoring data and improving the understanding of the sequestration process.

As an alternative to using fiberglass casing, resistivity antennas can be planted in the formation behind a metal casing to enable long-term monitoring of CO2 sequestration. These antennas, which are essentially electrodes embedded in the formation, can measure the resistivity of the surrounding rock and fluids, providing valuable information about changes in the formation due to CO2 injection and mineralization. Like the deep resistivity or dielectric logging tools used with fiberglass casing, these antennas can help track the movement of the CO2 plume and changes in the brine composition. However, unlike logging tools, which are typically run in the wellbore after the completion of drilling, these antennas can provide continuous, real-time monitoring data. This can provide a more detailed picture of the changes in the formation over time and can help detect any unexpected developments, such as leaks or rapid changes in the CO2 plume. Therefore, while the installation of resistivity antennas can be more complex and costly than using fiberglass casing, they can provide a powerful tool for monitoring CO2 sequestration.

Also as an alternative to using fiberglass monitoring, plume monitoring may be accomplished by acoustic means. Plume monitoring during CO2 sequestration can be effectively conducted using various acoustic logging techniques, including surface seismic surveys, borehole seismic measurements, and fiber distributed acoustic sensing (DAS). Surface seismic surveys involve generating seismic waves at the surface and recording their reflections and refractions to create a detailed image of the subsurface. By monitoring these seismic reflections and refractions, it is possible to identify and track the movement of the CO2 plume within the reservoir. This method provides a large-scale view of the plume distribution and can help assess its migration pathways.

Borehole seismic measurements involve deploying sensors in a wellbore to record seismic data at various depths. These measurements offer a more detailed and localized view of the plume behavior. By analyzing the seismic waves, their travel times, and their interactions with the formation, the extent and movement of the CO2 plume can be determined with higher resolution. Borehole seismic monitoring provides valuable insights into the vertical distribution and heterogeneity of the plume, facilitating a more precise understanding of the sequestration process.

Fiber distributed acoustic sensing (DAS) is a technique that utilizes fiber optic cables installed along the wellbore to measure acoustic signals. These signals are generated by the interaction of the CO2 plume with the surrounding formation. DAS allows for continuous monitoring of the plume's behavior and can provide valuable information on its spatial and temporal dynamics. By analyzing the acoustic signals, changes in amplitude, frequency, and arrival times can be detected, enabling the characterization and tracking of the CO2 plume.

Overall, plume monitoring through acoustic logging techniques, including surface seismic, borehole seismic, and fiber distributed acoustic sensing, offers valuable tools to assess the movement, distribution, and behavior of the CO2 plume during sequestration. These methods provide crucial insights into the effectiveness of the storage process and help evaluate the containment and migration of the injected CO2. By combining acoustic monitoring with other monitoring techniques, a comprehensive understanding of the plume behavior can be achieved, contributing to the overall success and safety of CO2 sequestration initiatives.

A well-planned sampling operation is of paramount importance when determining the brine chemistry during CO2 sequestration. Accurate and representative brine samples provide critical insights into the changes occurring in the reservoir due to CO2 injection and mineralization. Several considerations should be taken into account when designing the sampling plan. First, it is crucial to identify the appropriate sampling locations within the well to capture the spatial variability of the brine chemistry. This may involve selecting multiple depths and different regions along the wellbore. Additionally, the sampling frequency should be determined, considering factors such as the expected reaction rates and the desired temporal resolution of the data.

To ensure the reliability and integrity of the samples, proper sampling techniques should be employed. This includes utilizing specialized downhole samplers or wireline tools that can collect representative fluid samples at the desired depths. Care should be taken to avoid contamination during sample collection, handling, and storage to maintain the integrity of the brine chemistry. In some embodiments, brines may be flowed to the surface through a monitor well. Furthermore, it is important to consider the analysis methods for determining the brine chemistry. This may involve conducting laboratory tests such as ion chromatography, spectrophotometry, or mass spectrometry to measure the concentrations of various ions, pH, alkalinity, and other relevant parameters. The selected analysis methods should be accurate, precise, and capable of detecting small changes in the brine chemistry associated with CO2 mineralization.

The sampling plan should also consider the potential impact of well operations, such as CO2 injection, on the brine chemistry. These operations may cause transient changes in the composition and properties of the brine, which should be accounted for when planning the sampling intervals and interpreting the data.

Overall, a well-planned sampling operation allows for the comprehensive understanding of the brine chemistry and its changes over time, providing crucial data to assess the progress and effectiveness of CO2 sequestration. It enables the quantification of the reaction extent, identification of mineral trapping, and verification of the stability of the sequestered CO2, ultimately supporting the proof of successful carbon storage and sequestration.

The combination of various monitoring techniques, laboratory experiments, and data analysis methods discussed above forms a comprehensive strategy to effectively monitor CO2 sequestration. By integrating these elements, a holistic understanding of the sequestration process can be achieved, providing valuable insights into the extent and effectiveness of carbon storage.

The laboratory experiments conducted on core samples allow for controlled investigations of the mineralization reactions and provide fundamental data on reaction kinetics, heat production, and changes in mineralogy. These experiments, coupled with modeling approaches, enable the prediction of reaction rates and reaction extents under reservoir conditions. The data obtained from laboratory experiments serve as a foundation for interpreting the field monitoring data and validating the predictive models. By analyzing the mineral content of the cores before and after CO2 exposure, insights can be made into the specific reactions that are occurring and how they contribute to overall CO2 sequestration:

Initial Mineral Analysis: Use techniques like X-ray diffraction (XRD) or energy-dispersive X-ray spectroscopy (EDS) to quantify the mineral content of the cores before CO2 exposure. This will give a baseline against which can be compared to the post-exposure mineralogy.

CO2 Exposure and Monitoring: Expose the cores to CO2 under conditions that simulate those in the formation. Monitor the system over time to track changes in the fluid composition, pH, and other parameters that could indicate mineral reactions.

Final Mineral Analysis: After the CO2 exposure period, analyze the mineral content of the cores again. Look for changes in the quantities of specific minerals that could indicate CO2 sequestration. For example, a decrease in silicate minerals and an increase in carbonate minerals could suggest that CO2 is being sequestered through mineral carbonation reactions.

Correlation and Modeling: Correlate the changes in mineral content with the observed reaction dynamics. This could involve statistical analysis or more complex geochemical modeling. The goal is to identify which minerals are most reactive and how they contribute to CO2 sequestration.

Field monitoring techniques, such as fiber optic temperature monitoring, resistivity logging, and NMR logging, provide real-time and spatially distributed information on the changes occurring within the reservoir. These techniques help track the movement of the CO2 plume, identify changes in brine composition, monitor heat production, and assess alterations in the physical properties of the formation. The integration of these field monitoring data with the laboratory results allows for a more comprehensive understanding of the sequestration process and the verification of the desired reactions.

Having a comprehensive temperature profile of the reservoir may be critical. This data allows the monitor the spatial distribution of heat in the reservoir, which can provide insights into the locations and extents of CO2 mineralization reactions:

Heat Flow Modeling: With temperature data from multiple points in the reservoir, a can refined heat flow model may be. This model can help interpret the temperature data and estimate the amount of heat produced by the CO2 mineralization reactions.

Reaction Kinetics: The temperature data can also provide information on the kinetics of the CO2 mineralization reactions. These reactions are likely to be temperature-dependent, so by monitoring the temperature, insights can be gained into the reaction rates.

Reservoir Management: The temperature data can also be useful for managing the CO2 injection process. For example, if certain areas of the reservoir are heating up more than others, it might indicate that more CO2 is being sequestered in those areas. This information could be used to adjust the CO2 injection strategy to maximize sequestration.

Long-term Monitoring: Over the long term, the temperature data can help monitor the stability of the sequestered CO2. If the temperature in the reservoir starts to increase or decrease unexpectedly, it could indicate a change in the state of the sequestered CO2.

Monitoring changes in surface roughness, porosity, and permeability of the rock, as well as resistivity changes in the brine, can provide valuable insights into the extent of CO2 mineralization:

Surface Roughness: Changes in surface roughness could indicate mineralization reactions occurring on the rock surface. These changes could be monitored using various logging tools, such as micro-resistivity imagers, that can provide high-resolution images of the borehole wall.

Porosity and Permeability: Changes in porosity and permeability could indicate the formation of new minerals within the pore spaces of the rock. These changes could be monitored using various logging tools, such as nuclear magnetic resonance (NMR) or sonic logs, that can provide information on porosity and permeability.

Resistivity Changes: Changes in the resistivity of the brine could indicate changes in its composition due to CO2 mineralization reactions. These changes could be monitored using resistivity logging tools. The resistivity contrast between the brine and CO2 can also help track the movement of the CO2 injection plume.

Dielectric Properties: Changes in the dielectric properties of the fluids could also indicate changes in their composition or the movement of the CO2 plume. These changes could be monitored using dielectric logging tools.

Thermal Monitoring: Thermal monitoring can provide additional information on the extent of the CO2 mineralization reactions. The heat produced by these reactions can be monitored using fiber optic cables.

Correlation with Laboratory Studies: The downhole monitoring data can be correlated with the results of the laboratory studies on core samples. This can help validate the laboratory results and improve the understanding of the CO2 mineralization process in the formation.

Resistivity logging tools can provide information about the formation several tens to hundreds of feet away from the wellbore, depending on the specific tool and formation properties. This can be particularly useful for monitoring changes in the formation over a larger area and for tracking the movement of the CO2 injection plume.

Resolution and Sensitivity: While resistivity tools can provide information over a larger area, their resolution and sensitivity typically decrease with distance from the wellbore. So, the data from farther away from the wellbore may be less detailed and more influenced by larger-scale trends in the formation.

Interpretation: Interpreting resistivity data can be complex, as resistivity can be affected by various factors, including the porosity, permeability, and mineralogy of the formation, as well as the composition and temperature of the fluids. So, it's important to take these factors into account when interpreting the resistivity data.

Integration with Other Data: Resistivity data can be most informative when integrated with other data, such as temperature, pressure, and fluid composition data. This can help better understand the changes in the formation due to CO2 injection and mineralization.

Modeling: Resistivity data can also be used to update and validate the models of the CO2 injection and mineralization process. By comparing the observed resistivity changes with the predictions of the models, can gain insights into the accuracy of the models and potentially improve them.

Additionally, the analysis of brine chemistry data obtained through well-planned sampling operations provides essential insights into the changes in fluid composition due to CO2 injection and mineralization. By tracking the variations in ion concentrations, pH, and alkalinity, the effectiveness of CO2 sequestration can be assessed, and the stability of the sequestered carbon can be verified. These measurements, combined with the data from other monitoring techniques, contribute to a holistic assessment of the overall success of the sequestration strategy.

Analyzing the composition of the produced brines over time can provide valuable insights into the CO2 mineralization reactions. As CO2 reacts with the minerals in the formation, it can cause changes in the concentrations of various ions in the brine:

Baseline Analysis: Before CO2 injection begins, analyze the composition of the brine to establish a baseline. This should include measurements of pH, alkalinity, and the concentrations of major ions (e.g., Ca2+, Mg2+, Fe2+, HCO3−, SO42−, Cl−).

Periodic Sampling: During and after CO2 injection, periodically sample the produced brine and analyze its composition. Look for changes in the concentrations of specific ions that could indicate CO2 mineralization reactions. For example, a decrease in Ca2+ or Mg2+ and an increase in HCO3− could suggest the formation of carbonate minerals.

Correlation with Temperature Data: Compare the changes in brine composition with the temperature data. If certain ions are increasing or decreasing in concentration at the same time that the temperature is changing, it could indicate that those ions are involved in exothermic or endothermic reactions with CO2.

Geochemical Modeling: Use geochemical modeling to interpret the brine composition data. The model can help identify the most likely mineralization reactions and estimate the amount of CO2 sequestered.

Long-term Monitoring: Continue to monitor the brine composition over the long term to track the progress of CO2 sequestration. If the concentrations of certain ions start to level off or change direction, it could indicate that the CO2 mineralization reactions are slowing down or that new reactions are starting.

It is, the strategic combination of laboratory experiments, field monitoring techniques, and brine chemistry analysis provides a comprehensive framework for monitoring CO2 sequestration. This integrated approach allows for the verification of mineral trapping, quantification of reaction extents, assessment of heat production, and validation of predictive models. By leveraging these elements, scientists and engineers can gain a deep understanding of the sequestration process and provide reliable evidence to support the successful implementation of carbon storage and sequestration initiatives.

In this embodiment contains various novel aspects:

Use of temperature to monitor the temperature distribution of a wellbore heat change over time in order to determine the reaction extent of a CO2 mineralization process using a mineralization heat production model. The temperature may be determined using fiber optics or point temperature sensors. A mineralization heat production model may be a thermodynamic model. the model may be developed with laboratory CO2 mineralization studies. The model may be developed with core data from the field of interest, where core data is a proxy to the formation of interest.

Mineral distributions in core data may be used to develop a multi factor thermodynamic model. Such models may make evaluations of brine from a reservoir and brine data may be used to constrain temperature data. In such instances, brine data may come from sampling or from resistivity data. This resistivity data may be collected from fiberglass cased wells are used. The resistivity data may be collected from sensors on an outside surface of a casing. A heat flow model may be developed from laboratory core test data. The heat flow model may be constrained by thermal isolation in a wellbore in such instances, the thermal isolation may come from use of a fiberglass casing. The heat flow model may be constrained by the knowledge of CO2 plume distribution in the formation. One or more of permeability, porosity, or surface roughness data may be used to constrain the heat flow distribution model.

When brine data is used to monitor the extent of a CO2 mineralization reaction, the brine data may come from samples taken from a wellbore or from collected resistivity data. Modeling brine fluid changes may be based on laboratory core test data. In such instances, at least one of permeability, porosity, or surface roughness may be used to constrain the brine model.

Mineralization of a reservoir by CO2 may be inferred from at least one of permeability, porosity or surface roughness changes according to a baseline. In such instances, the model may be constrained by brine information, by thermal information, or by a distribution of a CO2 plume. Here again the CO2 plume may be monitored using resistivity data.

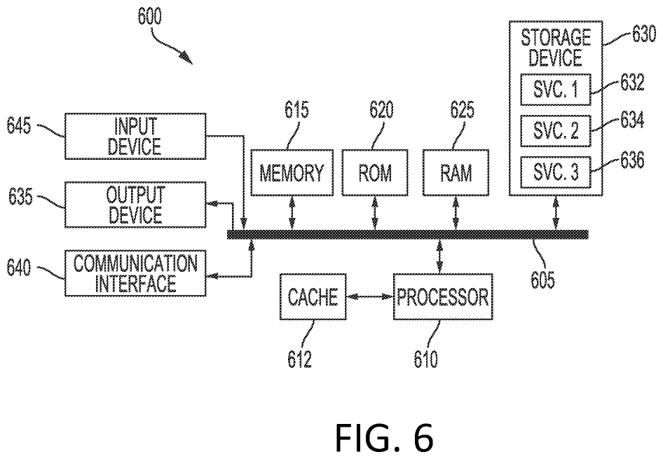

FIG. 6 illustrates an example computing device architecture 600 which can be employed to perform various steps, methods, and techniques disclosed herein. Specifically, the computing device architecture can be integrated with the electromagnetic imager tools described herein. Further, the computing device can be configured to implement the techniques of controlling borehole image blending through machine learning described herein.

As noted above, FIG. 6 illustrates an example computing device architecture 600 of a computing device which can implement the various technologies and techniques described herein. The components of the computing device architecture 600 are shown in electrical communication with each other using a connection 605, such as a bus. The example computing device architecture 600 includes a processing unit (CPU or processor) 610 and a computing device connection 605 that couples various computing device components including the computing device memory 615, such as read only memory (ROM) 620 and random access memory (RAM) 625, to the processor 610.

The computing device architecture 600 can include a cache of high-speed memory connected directly with, in close proximity to, or integrated as part of the processor 610. The computing device architecture 600 can copy data from the memory 615 and/or the storage device 630 to the cache 612 for quick access by the processor 610. In this way, the cache can provide a performance boost that avoids processor 610 delays while waiting for data. These and other modules can control or be configured to control the processor 610 to perform various actions. Other computing device memory 615 may be available for use as well. The memory 615 can include multiple different types of memory with different performance characteristics. The processor 610 can include any general purpose processor and a hardware or software service, such as service 1 632, service 2 634, and service 3 636 stored in storage device 630, configured to control the processor 610 as well as a special-purpose processor where software instructions are incorporated into the processor design. The processor 610 may be a self-contained system, containing multiple cores or processors, a bus, memory controller, cache, etc. A multi-core processor may be symmetric or asymmetric.

To enable user interaction with the computing device architecture 600, an input device 645 can represent any number of input mechanisms, such as a microphone for speech, a touch-sensitive screen for gesture or graphical input, keyboard, mouse, motion input, speech and so forth. An output device 635 can also be one or more of a number of output mechanisms known to those of skill in the art, such as a display, projector, television, speaker device, etc. In some instances, multimodal computing devices can enable a user to provide multiple types of input to communicate with the computing device architecture 600. The communications interface 640 can generally govern and manage the user input and computing device output. There is no restriction on operating on any particular hardware arrangement and therefore the basic features here may easily be substituted for improved hardware or firmware arrangements as they are developed.

Storage device 630 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 625, read only memory (ROM) 620, and hybrids thereof. The storage device 630 can include services 632, 634, 636 for controlling the processor 610. Other hardware or software modules are contemplated. The storage device 630 can be connected to the computing device connection 605. In one aspect, a hardware module that performs a particular function can include the software component stored in a computer-readable medium in connection with the necessary hardware components, such as the processor 610, connection 605, output device 635, and so forth, to carry out the function.

For clarity of explanation, in some instances the present technology may be presented as including individual functional blocks including functional blocks comprising devices, device components, steps or routines in a method embodied in software, or combinations of hardware and software.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can include hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the disclosed concepts may be otherwise variously embodied and employed, and that the appended claims are intended to be construed to include such variations, except as limited by the prior art. Various features and aspects of the above-described subject matter may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the method, algorithms, and/or operations described above. The computer-readable data storage medium may form part of a computer program product, which may include packaging materials.

The computer-readable medium may include memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

In the above description, terms such as “upper,” “upward,” “lower,” “downward,” “above,” “below,” “downhole,” “uphole,” “longitudinal,” “lateral,” and the like, as used herein, shall mean in relation to the bottom or furthest extent of the surrounding wellbore even though the wellbore or portions of it may be deviated or horizontal. Correspondingly, the transverse, axial, lateral, longitudinal, radial, etc., orientations shall mean orientations relative to the orientation of the wellbore or tool. Additionally, the illustrate embodiments are illustrated such that the orientation is such that the right-hand side is downhole compared to the left-hand side.

The term “coupled” is defined as connected, whether directly or indirectly through intervening components, and is not necessarily limited to physical connections. The connection can be such that the objects are permanently connected or releasably connected. The term “outside” refers to a region that is beyond the outermost confines of a physical object. The term “inside” indicates that at least a portion of a region is partially contained within a boundary formed by the object. The term “substantially” is defined to be essentially conforming to the particular dimension, shape or another word that substantially modifies, such that the component need not be exact. For example, substantially cylindrical means that the object resembles a cylinder, but can have one or more deviations from a true cylinder.

The term “radially” means substantially in a direction along a radius of the object, or having a directional component in a direction along a radius of the object, even if the object is not exactly circular or cylindrical. The term “axially” means substantially along a direction of the axis of the object. If not specified, the term axially is such that it refers to the longer axis of the object.

Although a variety of information was used to explain aspects within the scope of the appended claims, no limitation of the claims should be implied based on particular features or arrangements, as one of ordinary skill would be able to derive a wide variety of implementations. Further and although some subject matter may have been described in language specific to structural features and/or method steps, it is to be understood that the subject matter defined in the appended claims is not necessarily limited to these described features or acts. Such functionality can be distributed differently or performed in components other than those identified herein. The described features and steps are disclosed as possible components of systems and methods within the scope of the appended claims.

Moreover, claim language reciting “at least one of” a set indicates that one member of the set or multiple members of the set satisfy the claim. For example, claim language reciting “at least one of A and B” means A, B, or A and B.

What is claimed is:

1. A method comprising:
collecting a first set of wellbore data before a first mass of carbon dioxide (CO2) is injected into a wellbore;
injecting the first mass of the CO2 into the wellbore;
collecting a second set of wellbore data;
identifying a change associated with a formation surrounding the wellbore; and
estimating a quantity of the first mass of the CO2 that has been transformed into a mineral compound by a chemical reaction based on the identified change associated with the formation.

2. The method of claim 1, further comprising:
transmitting a first electromagnetic field into the formation while the first set of wellbore data is collected; and
transmitting a second electromagnetic field into the formation while the second set of wellbore data is collected.

3. The method of claim 1, further comprising:
transmitting a first acoustic signal into the materials of the formation while the first set of wellbore data is collected; and
transmitting a second acoustic signal into the materials of the formation while the second set of wellbore data is collected.

4. The method of claim 1, further comprising:
deploying one or more sensors along an inside surface of a casing of the wellbore, wherein the one or more sensors sense the first and the second set of wellbore data based on being deployed on the inside surface of the casing.

5. The method of claim 4, wherein the casing includes an electrical insulating material.

6. The method of claim 4, wherein the electrical insulating material of the casing resists corrosion and allows electromagnetic fields to propagate through the casing.

7. The method of claim 1, wherein the change associated with the materials of the Earth corresponds to a temperature difference associated with the injection of the first mass of the CO2 into the wellbore.

8. The method of claim 1, further comprising:
placing a rock sample into a pressure-temperature chamber;
heating the pressure-temperature chamber to a reference temperature that corresponds to a wellbore condition associated with the reference temperature and a reference pressure; and
providing a second mass of the CO2 to the chamber when a simulation is performed to estimate effects of the first mass of the CO2 being injected into the wellbore based on the wellbore condition associated with the reference temperature and the reference pressure.

9. The method of claim 8, further comprising:
collecting a third set of data before the second mass of the CO2 is provided to the chamber;
identifying a first value of mineralization associated with the sample based on an evaluation of the third set of data;
collecting a fourth set of data after the second mass of the CO2 is provided to the chamber;
identifying a second value of mineralization associated with the sample based on an evaluation of the fourth set of data; and
identifying a percentage of the second mass of CO2 that has been transformed into the mineral compound by the chemical reaction based on a difference between the second value of mineralization and the first value of mineralization.

10. The method of claim 9, further comprising:
updating a computer model based on the percentage of the second mass of CO2 that has been transformed into the mineral compound by the chemical reaction, wherein the estimated quantity of the first mass of the CO2 that has been transformed into the mineral compound by the chemical reaction is based on application of the updated computer model.

11. A non-transitory computer-readable storage media having embodied thereon instructions executable by one or more processors to implement a method comprising:
collecting a first set of wellbore data before a first mass of carbon dioxide (CO2) is injected into a wellbore;
controlling injection of the first mass of the CO2 into the wellbore;
collecting a second set of wellbore data;
identifying a change associated with a formation surrounding the wellbore; and
estimating a quantity of the first mass of the CO2 that has been transformed into a mineral compound by a chemical reaction based on the identified change associated with the formation.

12. The non-transitory computer-readable storage media of claim 11, wherein the one or more processors execute the instructions to:
initiate transmission of a first electromagnetic field into the formation while the first set of wellbore data is collected; and
initiate transmission of a second electromagnetic field into the formation while the second set of wellbore data is collected.

13. The non-transitory computer-readable storage media of claim 11, wherein the one or more processors execute the instructions to:
initiate transmission of a first acoustic signal into the materials of the formation while the first set of wellbore data is collected; and
initiate transmission of a second acoustic signal into the materials of the formation while the second set of wellbore data is collected.

14. The non-transitory computer-readable storage media of claim 11, wherein one or more sensors are deployed along an inside surface of a casing of the wellbore, and wherein the one or more sensors sense the first and the second set of wellbore data based on being deployed on the inside surface of the casing.

15. The non-transitory computer-readable storage media of claim 14, wherein the casing includes an electrical insulating material.

16. The non-transitory computer-readable storage media of claim 11, wherein the change associated with the materials of the Earth corresponds to a temperature difference associated with the injection of the first mass of the CO2 into the wellbore.

17. The non-transitory computer-readable storage media of claim 11, wherein:
a rock sample is placed into a pressure-temperature chamber;
the pressure-temperature chamber is heated to a reference temperature that corresponds to a wellbore condition associated with the reference temperature and a reference pressure; and
a second mass of the CO2 is provided to the chamber when a simulation is performed to estimate effects of the first mass of the CO2 being injected into the wellbore based on the wellbore condition associated with the reference temperature and the reference pressure.

18. The non-transitory computer-readable storage media of claim 17, wherein the one or more processors execute the instructions to:
collect a third set of data before the second mass of the CO2 is provided to the chamber;
identify a first value of mineralization associated with the sample based on an evaluation of the third set of data;
collect a fourth set of data after the second mass of the CO2 is provided to the chamber;
identify a second value of mineralization associated with the sample based on an evaluation of the fourth set of data; and
identify a percentage of the second mass of CO2 that has been transformed into the mineral compound by the chemical reaction based on a difference between the second value of mineralization and the first value of mineralization.

19. An apparatus comprising:
one or more sensors that collect a first set of wellbore data before a first mass of carbon dioxide (CO2) is injected into a wellbore;
a CO2 source that provides the first mass of the CO2 into the wellbore, wherein the one or more sensors collects a second set of wellbore data;
a memory; and
a processor that executes instructions out of the memory to:
identify a change associated with a formation surrounding the wellbore; and
estimate a quantity of the first mass of the CO2 that has been transformed into a mineral compound by a chemical reaction based on the identified change associated with the formation.

20. The apparatus of claim 19, further comprising:
one or more electromagnetic transmitters that:
transmit a first electromagnetic field into the formation while the first set of wellbore data is collected; and
transmit a second electromagnetic field into the formation while the second set of wellbore data is collected.
